(19) United States
(12) Patent Application Publication
(10) Pub. No.: US 2026/0140267 A1
(43) Pub. Date: May 21, 2026

(54) FLASH Radiation Therapy Dosimeter

(71) Applicant: Standard Imaging, Inc., Middleton, WI (US)

(72) Inventors: Brian Douglas Hooten, Middleton, WI (US); Shannon Mary Holmes, Solon, IA (US); Myles Lawton Sommerfeldt, Madison, WI (US); Raymond Terry Riddle, Middleton, WI (US); Larry Albert DeWerd, Fitchburg, WI (US)

(21) Appl. No.: 18/952,533

(22) Filed: Nov. 19, 2024

(51) Int. Cl. G01T 1/14; A61N 5/10

(52) U.S. Cl. CPC G01T 1/14; A61N 5/1071; A61N 2005/1074

(57) ABSTRACT

An ionization detector provides accurate measurements of high dose rate, short duration radiation pulses required by FLASH radiation therapy procedures by employing high voltages across narrow ionization channels minimizing saturation and polarization effects. Air equivalent polymers may be used to provide the electrodes of the ionization detector for improved accuracy and to resist radiation-induced degradation.

### STATEMENT REGARDING FEDERALLY SPONSORED RESEARCH OR DEVELOPMENT

### CROSS REFERENCE TO RELATED APPLICATION

### Background of the Invention

The present invention relates generally to FLASH radiotherapy, a medical treatment using high-dose, short duration radiation to treat tumors and the like, and in particular to a dosimeter for making accurate measurements of FLASH radiation dose and allowing calibration of such FLASH equipment.

Radiation therapy (RT) is a crucial component of curative cancer therapy, with about 50% of U.S. cancer patients receiving RT as part of their treatment. Key factors limiting current effectiveness of RT are acute and long-term normal tissue toxicities which often preclude delivery of curative radiation doses.

Pioneering preliminary work has demonstrated that ultra-rapid delivery of RT (dose rate >30 Gy/s), dubbed “FLASH,” spares normal tissues and organs selectively while maintaining tumor kill in in vivo preclinical models. This represents a fundamentally new prospect for increasing the therapeutic index of RT compared to the same doses given at conventional dose rates (0.1-0.2 Gy/s).

Although the FLASH effect has been observed in many organ systems, the physical irradiation parameters needed to achieve the effect are still largely unknown. Early evidence indicated a mean dose rate threshold of ˜40 Gy/s, but evidence is now accumulating for a dynamic mean dose rate dependence, in which increased dose rates induce higher normal tissue-sparing effects. Furthermore, the full effect of dose per pulse, pulse repetition frequency, and instantaneous dose rate on the magnitude of the FLASH sparing effect is so far unknown.

Important in the determination of the proper irradiation parameters is a method of accurately measuring such short duration, high-dose radiation pulses. Desirably the dosimeter for FLASH will have rapid response, good accuracy, and the ability to be calibrated at a secondary standard dosimetry laboratory. For conventional radiotherapy, these requirements are often met with a so-called ionization detector detecting ionization of gas between electrodes. Unfortunately, current ionization detectors failed to accurately measure FLASH dose because of saturation effects that cannot be accounted for using established correction factors.

### SUMMARY OF THE INVENTION

The present inventors have determined extremely narrow ionization gaps and high electric field strengths can significantly decrease inaccuracies in the measurement of FLASH radiotherapy pulses experienced with conventional ionization detectors. While the inventors do not wish to be bound by a particular theory, it is believed that inaccuracies of ionization detectors come from charge recombination and polarization in dense clouds of ionized gas which can be reduced by short distances and high voltages which quickly deplete the ions before significant recombination and polarization. In some embodiments, the present invention makes use of solid conductive polymers for electrodes addressing problems of radiation-induced degradation at high dose rates.

More specifically, the invention may provide an ionization detector suitable for use with FLASH radiation treatments and including a first and second electrode spaced from each other by a gap of less than 0.9 mm. A power supply or source is connected to the ionization detector to apply a voltage across the first and second electrodes sufficient to create an electric field strength greater than 500 Vdc/mm. A current sensor connected to the first and second electrodes measures charge passing between the first and second electrodes.

It is thus a feature of at least one embodiment of the invention to construct a fully guarded ionization type detector that can accurately measure short, high dose rates of a type employed in FLASH radiotherapy.

In some embodiments, the first, second, and third electrodes may be a conductive polymer providing an air equivalent radiation absorption.

It is thus a feature of at least one embodiment of the invention to minimize scattering artifacts at high dose rates and interference with the measurement caused by material absorption.

In some embodiments, the conductive polymer of the first and second electrodes may have a minimum thickness of greater than 0.1 mm.

It is thus a feature of at least one embodiment of the invention to make use of solid polymer conductors that have been determined to be robust against damage at FLASH dose rates.

The ionization detector system may further include a display communicating with the current sensor to provide an output indicating at least one of a measurement of current and charge.

It is thus a feature of at least one embodiment of the invention to provide an ionization detector presenting an output familiar to those experienced in using radiation dosimeters.

These particular objects and advantages may apply to only some embodiments falling within the claims and thus do not define the scope of the invention.

### BRIEF DESCRIPTION OF THE DRAWINGS

FIG. 1 is a simplified diagram of a FLASH radiation system showing positioning of the ionization detector of the present invention as connected to a measurement circuit for measurement of radiation dose;

FIG. 2 is a perspective view of the ionization detector of FIG. 1;

FIG. 3 is an exploded fragmentary view of a detector head of the ionization detector of FIG. 2; and

FIG. 4 is a cross-sectional elevational view in fragment along line 4-4 of FIG. 2 showing the internal construction of the ionization detector.

### DETAILED DESCRIPTION OF THE PREFERRED EMBODIMENT

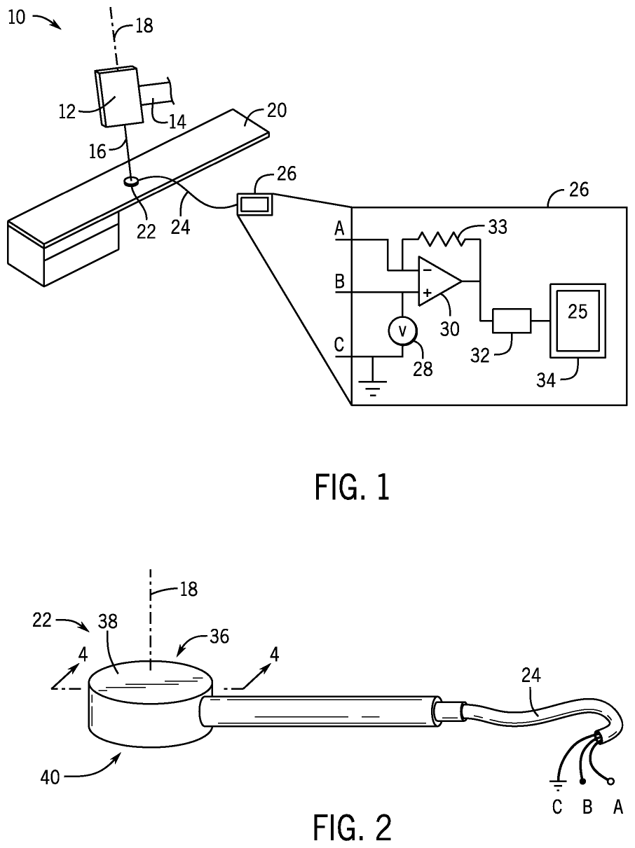

Referring now to FIG. 1, a FLASH radiation therapy system 10 may provide a radiation source 12, for example, capable of producing a radiation beam 16 directed along an axis 18 with a dose rate in the tens of Gy/s or more, for example, at 40 Gy/s. The radiation source 12 will typically be supported on a movable gantry arm 14 to orient the beam axis 18 toward a patient support 20 on which a patient may be positioned. An ionization detector 22 may be supported within the radiation beam 16 to measure the dose rate of the radiation beam 16 for calibration and the like.

Referring still to FIG. 1, the ionization detector 22 may be connected by a flexible cable 24 or the like to an electrometer 26 providing a quantitative display of the electrical current, charge, dose, and/or dose rate measured by the ionization detector 22 and to provide power to the ionization detector 22. The electrometer 26, in one nonlimiting example, may receive three conductors from the ionization detector 22, for example, being separate conductors of a triaxial cable forming the flexible cable 24 and here labeled: A, B, C. Conductor C may be a ground reference and connected to a ground terminal of a high-voltage DC power supply 28 capable of producing an electric field strength greater than 500 Vdc/mm and often in excess of 1000 Vdc/mm.

The power terminal of the power supply 28 may connect in turn to the conductor B which may also be connected to a noninverting input of a high-impedance instrumentation amplifier 30. Conductors A may connect to the inverting input of the instrumentation amplifier 30, and a feedback resistor 33 may shunt this inverting input and the output of the instrumentation amplifier 30 to provide an adjustable gain factor. Generally the output may permit measurement of a range of 0.001 pA to 500 nA or 0.0001 pC to 999.9 μC.

The output of the instrumentation amplifier 30 may be connected to a scaler circuit 32 applying a calibration factor or calibration curve to the output of the instrumentation amplifier 30 to permit a quantitative output on a digital display 34, for example, of dose calibrated in Grays or charge calibrated in Coulombs or current calibrated in Amperes. This calibration may be conducted on a unit by unit basis using accepted AAPM protocols and establishing absolute dose calibrations with NIST traceable detectors as is legally mandated in the U.S.

Electrometers 26 suitable for use with the present invention include those available from Standard Imaging, Inc. of Middleton, Wisconsin, under the tradenames of Super MAX and MAX Elite.

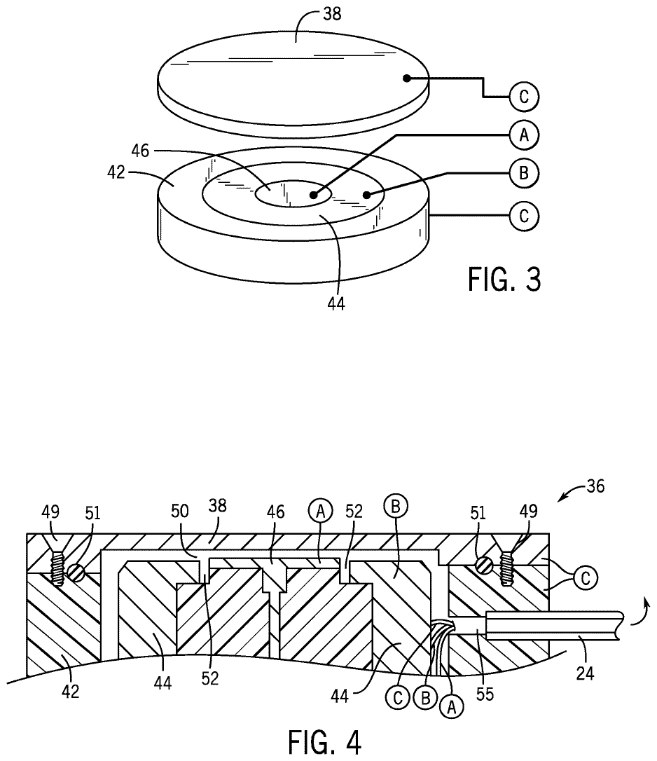

Referring now to FIGS. 2 and 3, the ionization detector 22 may include a head portion 36, for example, having a cylindrical body with a generally upper planar base 38 that may be aligned to be substantially perpendicular to the axis 18 of the radiation beam 16 during measurement. This orientation may be ensured by supporting the head portion 36 on its lower base 40 on the patient support 20 or the like or through a fixture (not shown) for that purpose.

The upper planar base 38 may attach to a cylindrical and tubular outer housing 42 forming the sidewalls of the base and holding concentrically and coaxially within the walls of the cylindrical tubular housing 42, but electrically isolated therefrom, a guard ring 44.

Arranged concentrically and coaxially within the guard ring 44, but electrically insulated therefrom, is a disk-shaped collector plate 46, for example, having a circular upper planar surface parallel to but positioned beneath, and electrically isolated from, a planar lower surface of the upper planar base 38.

Each of the components of the upper planar base 38, cylindrical tubular housing 42, guard ring 44, and collector plate 46 may be constructed of an electrically conductive polymer, for example, a carbon infused air-equivalent plastic such as Shonka C-552 having typical properties as follows and providing a radio translucency similar to that of air at the intended energies.

$$Density ( g / cm 3 ) = 1.7 \; 6000 E + 00$$

$$Mean Excitation Energy ( eV ) = 86.8$$

### Composition:

| Atomic | |
|---|---|
| number | Fraction by weight |
| 1 | 0.024680 |
| 6 | 0.501610 |
| 8 | 0.004527 |
| 9 | 0.465209 |
| 14 | 0.003973 |

Generally, each of the components receiving significant radiation during use will have a minimum thickness along the axis 18 or in any direction of greater than 0.1 mm and typically greater than 0.3 mm, these thicknesses of solid polymer demonstrated to provide survivability under exposure to high-dose radiation.

The upper planar base 38 and the housing 42 may be electrically interconnected to terminal C or ground, while the guard ring 44 is electrically connected to terminal B to be at a high-voltage potential with respect to the upper planar base 38. The collector plate 46 may be electrically connected to terminal A which will have a similar voltage to the guard ring 44 although electrically insulated therefrom.

Referring now to FIG. 4, when assembled, the head portion 36 provides a parallel plate ionization detector having a gap between a lower surface of the upper planar base 38 and the parallel upper planar surface of the collector plate 46, for example, spaced apart by a gap 50 of less than 0.9 mm and typically less than 0.5 mm and in some cases in a range between 0.1 and 0.5 mm and greater than 0.1 mm to maintain electrical isolation across this gap 50. Generally, the upper planar base 38 may be a disk shape having a diameter of at least 3 mm defining a collector area.

A gap 52 between the guard ring 44 and the outer periphery of the collector plate 46 may be greater than 0.1 mm or greater than 0.5 mm to maintain electrical isolation across the gap 52. The guard ring may extend for a distance of 4 mm or more radially from the gap 52 to the outer periphery of the guard ring.

The upper planar base 38 may be sealed against the housing 42 using O ring 51 compressed under the force of countersunk machine screws 49. The housing 42 may otherwise be a solid block of material so that the gap 50 may be isolated against water leakage into the head portion 36. An air vent passage 55 may be provided from the gap 50 through an opening in the housing 42 sealed to the outer sheath of the flexible cable 24 allowing air passage through the flexible cable 24 along with the conductors A, B, and C to a remote location, for example, at the electrometer 26 permitting the head 36 to be fully immersed in water during use.

It will be appreciated that this parallel plate structure can be implemented in a variety of different configurations including as attached to a printed circuit board or the like.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties

To aid the Patent Office and any readers of any patent issued on this application in interpreting the claims appended hereto, applicants wish to note that they do not intend any of the appended claims or claim elements to invoke 35 U.S.C. 112 (f) unless the words “means for” or “step for” are explicitly used in the particular claim.

What we claim is:

1. An ionization detector system suitable for use with FLASH radiation treatments comprising:
an ionization detector having:
(a) a first electrode; and
(b) a second electrode spaced from the first electrode by a gap of less than 0.9 mm;
a power supply applying a voltage generating an electric field greater than 500 Vdc/mm across the gap; and
a current sensor measuring charge passing between the first and second electrodes.

2. The ionization detector system of claim 1 wherein the first and second electrodes are a conductive polymer providing an air-equivalent radiation absorption.

3. The ionization detector system of claim 2 wherein the first and second electrodes are a conductive polymer and have a minimum irradiated thickness of greater than 0.1 mm.

4. The ionization detector system of claim 1 further including a display communicating with the current sensor to provide an output indicating at least one of a measurement of current or charge.

5. The ionization detector system of claim 1 wherein the first and second electrodes are parallel plates opposed along an axis about the gap and having a dimension perpendicular to the axis greater than 0.3 mm.

6. The ionization detector system of claim 1 further including a third electrode surrounding and coplanar with the second electrode and electrically biased at an offset voltage from the first electrode.

7. The ionization detector system of claim 6 wherein the ionization detector provides a watertight seal about the gap except through a vent connected to a flexible and elongated sheath to communicate with the outside air at a point remote from the ionization detector sufficient to allow immersion of the ionization detector in liquid during use.

8. The ionization detector system of claim 1 wherein the gap is between 0.1 and 0.5 mm·mm.

9. The ionization detector system of claim 1 wherein the electric field is greater than Vdc/mm.

10. A method of measuring radiation output from a FLASH radiation system using an ionization detector having a first electrode and a second electrode spaced from the first electrode by a gap of less than 0.9 mm, and having a power supply applying a voltage across the first and second electrodes sufficient to create an electric field greater than 500 Vdc/mm and a current sensor measuring charge passing between the first and second electrodes, the method comprising:
placing the ionization detector within a path of the FLASH radiation of greater than 30 Gy/s; and
measuring current passing between the first and second electrode to confirm dose or dose rate.

11. The method of claim 10 wherein the first and second electrodes are solid conductive polymers having a thickness of greater than 0.3 mm.

12. The method of claim 11 wherein the conductive polymer provides a water equivalent radiation absorption.

13. The method of claim 10 wherein the ionization detector further includes a third electrode surrounding and coplanar with the second electrode and electrically biased at an offset voltage from the first electrode.

14. The method of claim 10 further including converting measured current or charge into dose rate according to a conversion factor.

15. The method of claim 10 wherein the first and second electrodes are parallel plates opposed along an axis about the gap and having a dimension perpendicular to the axis of greater than 0.3 mm and including aligning the path of the radiation to pass normal to the parallel plates.

16. The method of claim 10 where in the ionization detector further includes a vent admitting outside air into the gap.

17. The method of claim 10 wherein the gap is between 0.1 and 0.5 mm.

18. The method of claim 10 wherein the electric field is greater than 1000 Vdc/mm.

19. The method of claim 10 further including the step of providing a NIST-traceable calibrated measurement of the FLASH machine output according to the dose or dose rate.

20. A FLASH radiation therapy system comprising:
a radiation source generating radiation pulses having a dose rate greater than 30 Gy/s;
an ionization detector positioned to receive radiation pulses and having:
(a) a first electrode; and
(b) a second electrode spaced from the first electrode by a gap of less than 0.9 mm mm;
(c) a third electrode surrounding and coplanar with the second electrode;
a power supply applying a voltage across the first and each of the second and third electrodes electrodes sufficient to produce an electric field greater than 500 Vdc/mm; and
a current sensor measuring a charge passing between the first and second electrodes.
